(19) United States
(12) Patent Application Publication
(10) Pub. No.: US 2024/0000330 A1
(43) Pub. Date: Jan. 4, 2024

(54) NANODEVICES AND METHODS FOR MEASURING BIOFLUIDIC FLOW USING A GRAPHENE-BASED MICROELECTRODE

(71) Applicant: University of Massachusetts, Boston, MA (US)

(72) Inventors: Jinglei Ping, Amherst, MA (US); Xiaoyu Zhang, Amherst, MA (US)

(21) Appl. No.: 18/081,604

(22) Filed: Dec. 14, 2022

Related U.S. Application Data
(60) Provisional application No. 63/311,123, filed on Feb. 17, 2022.

(51) Int. Cl. A61B 5/026; A61B 5/00

(52) U.S. Cl. CPC A61B 5/026; A61B 5/6847; A61B 2562/0285; A61B 2562/12

(57) ABSTRACT

The invention provides devices and methods for measuring microfluidic flow velocity. The novel electrical nanodevice employs a single microelectrode of monolayer graphene and measures in real time at high resolution and stability microfluidic flow velocity by quantifying contact electrification-induced current variations.

### PRIORITY CLAIMS AND RELATED PATENT APPLICATIONS

This application claims the benefit of priority to U.S. Provisional Application Ser. No. 63/311,123, filed on Feb. 17, 2022, the entire content of which is incorporated herein by reference in its entirety.

### STATEMENT REGARDING FEDERALLY FUNDED RESEARCH OR DEVELOPMENT

This invention was made with government support under Grant No. W81XWH-19-1-0006 awarded by Army Medical Research and Material Command and Grant No. FA9550-20-1-0125 awarded by Air Force Office of Scientific Research. The Government has certain rights in the invention.

### TECHNICAL FIELD OF THE INVENTION

The invention generally relates to devices and methods for flow-velocity measurements. More particularly, the novel electrical nanodevice disclosed herein employs a single microelectrode of monolayer graphene and measures the microfluidic flow velocity by quantifying contact electrification-induced current variations in real time at high resolution and stability.

### BACKGROUND OF THE INVENTION

Electrical devices employing nanomaterials and nanostructures have demonstrated high biocompatibility and miniaturizability and showed great promise in interfacing biosystems and measuring biofluidic flow at high spatiotemporal resolution. Conventional biofluid nanosensors, however, suffer from low sensitivity due to their planar structures. Bundled nanostructures may enhance the sensitivity but are prone to biofouling and clogging, making them unsuitable for continuous use in monitoring of complex biofluid. (Bourlon, et al. “A nanoscale probe for fluidic and ionic transport.” Nature Nanotechnology 2, 104 (2007); He et al. “Solution-gated graphene field effect transistors integrated in microfluidic systems and used for flow velocity detection” Nano Letters 12, 1404-1409 (2012); Newaz, et al. “Graphene transistor as a probe for streaming potential.” Nano Letters 12, 2931-2935 (2012); Sando, et al. in 2017 19th International Conference on Solid-State Sensors, Actuators and Microsystems (TRANSDUCERS). (IEEE, 2017), pp. 1738-1741; Zhang, et al. “Highly sensitive microfluidic flow sensor based on aligned piezoelectric poly (vinylidene fluoride-trifluoroethylene) nanofibers.” Applied Physics Letters 107, 242901 (2015); Bogomolova, et al. “Challenges of electrochemical impedance spectroscopy in protein biosensing.” Analytical Chemistry 81, 3944-3949 (2009); Ghosh, et al. “Carbon nanotube flow sensors.” Science 299, 1042-1044 (2003).)

There is an ongoing need for improved biofluid monitoring devices, in particular that can exhibit long-term stability, are immune to biofouling, and provide quasi-instant, non-perturbative and continuous flow monitoring.

### SUMMARY OF THE INVENTION

The invention provides a quasi-instant, non-perturbative flow meter based on graphene microsheet, which is immune to biofouling of non-specific chemicals and biomolecules. This flow meter does not require the application of gate voltage or source-drain bias, and demonstrates in real-time fluid flow with sensitivity that is suitable for in vivo monitoring.

In one aspect, the invention generally relates to an electrical nanodevice having a single microelectrode, comprising a monolayer graphene sheet serving as the single microelectrode disposed across a microfluidic channel.

In another aspect, the invention generally relates to a microfluidic flow sensor comprising an electrical nanodevice disclosed herein.

In yet another aspect, the invention generally relates to an implantable blood flow monitor comprising an electrical nanodevice disclosed herein.

In yet another aspect, the invention generally relates to a method for measuring a microfluidic flow velocity, comprising measuring a hydrovoltaic current variation arising from contact electrification between a monolayer graphene sheet and a microfluidic flow.

In yet another aspect, the invention generally relates to a method of fabricating an electrical nanodevice. The method comprises: providing a monolayer graphene sheet on a substrate; depositing a gold connection contact on a part of the monolayer graphene sheet; providing a microfluidic channel with an inlet and an outlet; and bonding the microfluidic channel with the monolayer graphene sheet.

### BRIEF DESCRIPTION OF THE DRAWINGS

FIG. 1. Schematic of device setup and test of blood which mimics human capillary. (A) Schematic of the electronic setup for measurement of Faradaic charge transfer between blood and graphene. The image shows a graphene microsheet in a microfluidic channel. The scale bar is 200 μm. (B) The flow quantitated by measuring the Faradaic charge transfer of the graphene microsheet and the syringe pump infusion flow, which mimics that for blood flow in human capillary, set by the syringe pump.

FIG. 2. Flow measurement of PBS. (A) Real-time charge transfer between graphene and full phosphate buffered saline (PBS) solution measured by the electrometer. (B) Faradaic current as a function of flow in PBS and bovine blood. The dashed lines are linear fittings. The inset shows the current response in log-log scale. (C) Upper panel: current response of the graphene microsheet to continuous flow ramps set by the syringe pump. Lower panel: comparison of the flow quantitated by measuring the Faradaic charge transfer of the graphene microsheet and the flow ramps.

FIG. 3. Flow measurement of bovine blood. (A) Upper panel: Real-time Faradaic current response of the graphene microsheet to the pump speed that mimic the flow in human arteriole. (B) Long-term Faradaic current response to 0.033, 3 and 6 mm/s pump velocity of bovine blood.

FIG. 4. The workflow of the device-fabrication process and as-fabricated flexible devices.

FIG. 5. The charge transfer of graphene is minimally associated to the graphene edge electronic states. (a) The charge-transfer current that was measured by using a 1-mm wide sheet of graphene (with two edges) and by using graphene stripes (with 16 edges) that were made from the graphene sheet by precision cutting. The syringe pump speed was changed in turn between 0.033, 4, and 8 mm s⁻¹. (b) The creation of the edges neither increased the calibration current magnitude nor the derivative of the current response with respect to flow velocity (the sensitivity). (c) The optical image of the graphene sheet prior to being cut. (d) The optical image of the graphene strips that were precision cut from the graphene sheet. The scale bars in c and d are 200 μm. The results indicated that the charge-transfer current and the current-flow association arose from the graphene basal plane electronic states.

FIG. 6. The measured unsmoothed charge transfer of a graphene device as a function of time for different blood-flow velocities. The diagram illustrates the electrical potential variation at a graphene-defect site and the corresponding variation in the electron transfer through the defect state, when the blood-flow velocity magnitude (v) changes. The vertical dash line indicates the graphene-defect state at the graphene/blood interface. The arrows represent the directions of the net electron flow.

FIG. 7. The monitored flow velocity in response to sawtooth-like blood-flow waveform driven by the syringe pump.

### DETAILED DESCRIPTION OF THE INVENTION

The invention is based in part on the discovery of a quasi-instant, non-perturbative flow meter based on graphene microsheet, which is immune to biofouling of non-specific chemicals and biomolecules. This flow meter does not require the application of gate voltage or source-drain bias, and demonstrates in real-time phosphate buffered saline (PBS) flow with sensitivity that is suitable for in vivo monitoring. The device may be used to study and uncover new biological and physiological phenomena such as metabolic state and central nervous system disorders. By measuring a graphene microsheet using a gate-free coulometric methodology, in vitro probing of real-time biofluid flow with the sensitivity of 30 μM/s was demonstrated. The unconventional approach disclosed herein paves the way for new meter-on-a-chip devices for chronic in vivo body fluid monitoring.

Applications that may benefit from the disclosed devices and methods include fast and ultra-low-power flow rate sensing of biofluids and measurement of small flow rate changes (30 μm/s). The flow meter has adequate sensitivity for real-time and in vivo monitoring of the flow rate of blood and sensing blood flow in human capillary that may not be detected by traditional flow meters.

The graphene-based flow meter disclosed herein is safe to use, fast to read out and immune to biofouling. It causes minimum perturbation and requires ultra-low power consumption. Unlike conventional graphene field-effect transistors (FETs), the graphene microsheet characterizes the flow rate through charge measurement rather than gate-voltage sweeping. The device does not require source-drain bias and is free of Johnson noise because electrical charges are stored in a feedback capacitor. The device is also immune to shot noise because random pulses are integrated, which affords higher sensitivity. Compared with bundled nanostructured flow meter, graphene microsheets are immune to biofouling and clogging, making them suitable for continuous use in monitoring of complex biofluid.

Thus, disclosed herein is a gate-free coulometric methodology that is employed to measure a graphene microsheet. A sensitivity of 30 μm/s was readily achieved in in vitro probing of real-time whole-blood flow.

In one aspect, the invention generally relates to an electrical nanodevice having a single microelectrode, comprising a monolayer graphene sheet serving as the single microelectrode disposed across a microfluidic channel.

In certain embodiments of the electrical nanodevice, the single microelectrode comprises a Au electrode deposited on a part of the monolayer graphene sheet for electrical connection.

In certain embodiments of the electrical nanodevice, the single microelectrode comprises a Cr/Au electrode deposited on a part of the monolayer graphene sheet for electrical connection.

In certain embodiments, the electrical nanodevice further comprises an operation amplifier having a feedback capacitor.

In certain embodiments, the microfluidic channel is defined on an acrylic sheet using laser cutting and bonded to the graphene sheet.

In certain embodiments, the monolayer graphene sheet is fabricated with a poly(methyl methacrylate) (PMMA) polymer layer.

In certain embodiments, the monolayer graphene sheet has a dimension in the range of about 1 μm×1 μm to about 1 mm×1 mm (e.g., 1 μm×1 μm to about 10 μm×10 μm, 10 μm×μm to about 100 μm×100 μm, 100 μm×100 μm to about 1 mm×1 mm).

In certain embodiments, the microfluidic channel has a dimension or cross-section in the range of about 1 μm×1 μm to about 1 mm×1 mm 1 μm×1 μm to about 10 μm×10 μm, 10 μm×10 μm to about 100 μm×100 μm, 100 μm×100 μm to about 1 mm×1 mm).

In certain embodiments, the electrical nanodevice does not comprise an external electrical supply.

In certain embodiments, the electrical nanodevice is biocompatible.

In certain embodiments, the electrical nanodevice is implantable.

In another aspect, the invention generally relates to a microfluidic flow sensor comprising an electrical nanodevice disclosed herein.

In yet another aspect, the invention generally relates to an implantable blood flow monitor comprising an electrical nanodevice disclosed herein.

In yet another aspect, the invention generally relates to a method for measuring a microfluidic flow velocity, comprising measuring a hydrovoltaic current variation arising from contact electrification between a monolayer graphene sheet and a microfluidic flow.

In certain embodiments of the method, the hydrovoltaic current variation is measured in vivo via an implanted electrical nanodevice.

In certain embodiments, the implanted electrical nanodevice does not comprise an external electrical supply.

In certain embodiments, the microfluidic flow is that of a biofluid.

In certain embodiments, the biofluid is whole blood, urine, saliva, cerebrospinal fluid, or serum.

In certain embodiments, the method is capable of measuring the microfluidic flow velocity characterized by variations at the μm/s level.

In certain embodiments, the method is capable of providing in vivo chronic body fluidic monitoring.

In yet another aspect, the invention generally relates to a method of fabricating an electrical nanodevice. The method comprises: providing a monolayer graphene sheet on a substrate; depositing a gold connection contact on a part of the monolayer graphene sheet; providing a microfluidic channel with an inlet and an outlet; and bonding the microfluidic channel with the monolayer graphene sheet.

In certain embodiments of the method, the monolayer graphene sheet is provided on a PMMA polymer layer.

In certain embodiments of the method, the microfluidic channel is defined using laser cutting.

In certain embodiments of the method, bonding is performed using an adhesive.

In certain embodiments, the method further comprises bonding a stainless-steel needle to the inlet and/or a stainless-steel needle to the outlet.

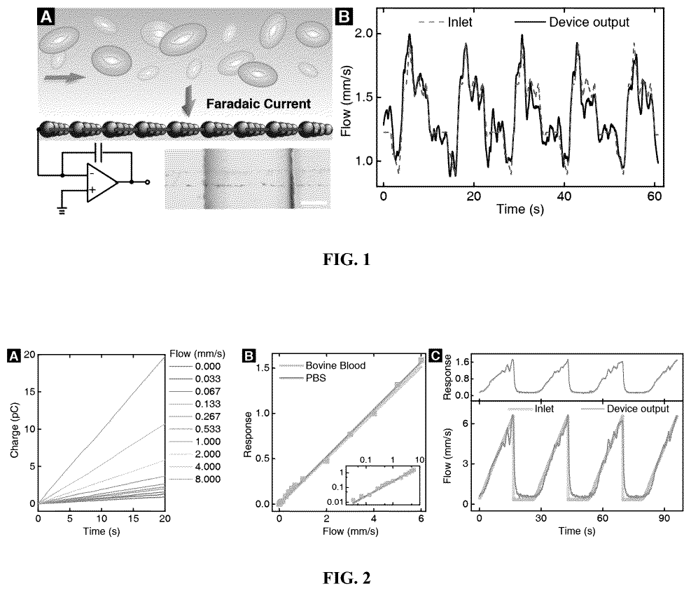

Without wishing to be bound by the theory, the sensitivity of electrical and electrochemical flow sensors is limited by two major factors: the Johnson noise of electrical current passing through a liquid gate and the non-specific electrochemical processes, e.g. physicochemical adsorptions, upon fouling at the liquid gate electrode by various components in the sample. It has now been demonstrated that at graphene-solution interfaces there is spontaneous Faradaic charge transfer, as shown in FIG. 1A. (Bogomolova, et al. “Challenges of electrochemical impedance spectroscopy in protein biosensing.” Analytical Chemistry 81, 3944-3949 (2009); Brezinski, “Kinetic, static and stirring errors of liquid junction reference electrodes.” Analyst 108, 425-442 (1983); Elzanowska, et al. “Reversible ageing of iridium oxide electrodes in acidic solutions.” Journal of Applied Electrochemistry 23, 646-654 (1992); Shinwari, et al. “Microfabricated reference electrodes and their biosensing applications.” Sensors 10, 1679-1715 (2010); Ping, et al. “Quantifying the intrinsic surface charge density and charge-transfer resistance of the graphene-solution interface through bias-free low-level charge measurement.” Applied Physics Letters 109, 013103 (2016).)

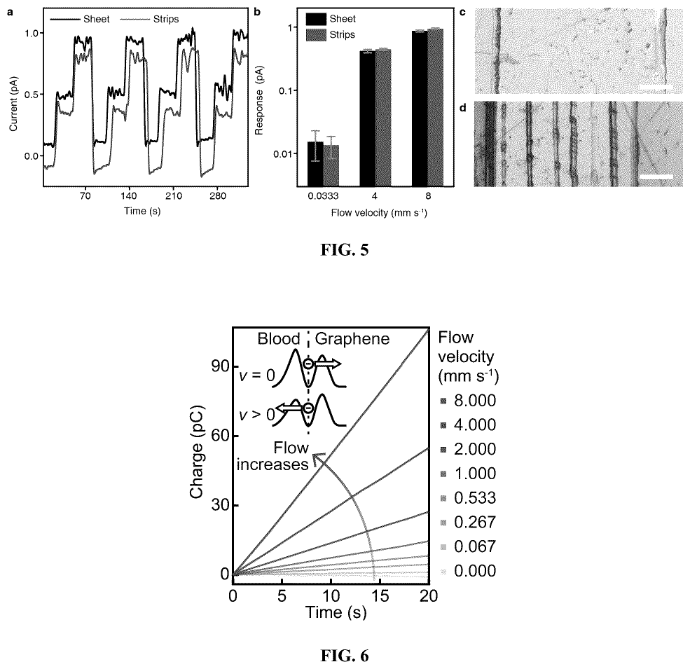

Due to the nature of covalent bonding and the low density of states (around Dirac point) of graphene, the charge transfer rate at graphene-solution interface is extremely low: about 10⁻⁵ cm-s⁻¹. Therefore, the interface is at perfect non-Faradaic limit and can be described by capacitor-like electrical double layer (EDL), which is formed by two layers of ions with opposite polarities, with finite “leaking” Faradaic charge transfer through graphene disorder. It is a long-term question about whether this charge transfer originates from in-plane disorder, edge states, or both. It has now been proven that the charge-transfer resistance R_{ct} of graphene is inversely proportional to the area ([L²]) instead of the length ([L]). It has also been demonstrated that the creation of edge state by slicing does not increase the Faradaic current correspondingly (FIG. 5). (Ping, et al. “Quantifying the intrinsic surface charge density and charge-transfer resistance of the graphene-solution interface through bias-free low-level charge measurement.” Applied Physics Letters 109, 013103 (2016); McCreery, et al. “Control of reactivity at carbon electrode surfaces.” Colloids and Surfaces A: Physicochemical and Engineering Aspects 93, 211-219 (1994); Yamada, et al. “Role of edge orientation in kinetics of electrochemical intercalation of lithium-ion at graphite.” Langmuir 26, 14990-14994 (2010); McCreery, et al. “Advance carbon electrode materials for molecular electrochemistry.” Chemical Reviews 108, 2646-2687 (2008).)

Thus, the Faradaic charge transfer arise from in-plane disorder of the graphene microsheet. Since the diffusion time constant for a charge to transfer from inside the solution through Nernst layer onto the graphene, about 0.1-1.0 s, is orders of magnitude less than the time constant for the charge to transfer into the graphene (1000 s), the electrostatic and electrochemical condition at the interfaces is quasi-static, which leads to time-invariant charge-transfer rate and self-driven Faradaic current. The current is characteristic of electrochemical potential at material-solution interfaces and is unique to graphene with all-covalent bonds and high chemical inertness at basal plane. (Ping, et al. “All-electronic quantification of neuropeptide-receptor interaction using a bias-free functionalized graphene microelectrode.” ACS Nano 12, 4218-4223 (2018).)

Furthermore, due to the atomic thickness of graphene, the microsheet placed with basal plane tangential to the follow is subject to shear force generated by the flow. The self-driven Faradaic current was quantitated and found that it is linearly related to the flow rate of the biofluid over orders of magnitude. Using a graphene microsheet in a microfluidic channel, the flow of whole bovine blood (BIOIVT BOV7411) was measured.

As shown in FIG. 1B, the quantitated blood speed agrees very well with the syringe pump (SyringePump No. 4000) dB. For each flow, the charge transfer between PBS solution and graphene microsheet increased linearly with time. The slope was used to determine the Faradaic current response in less than 20 s here. The limit of detection of the flow was less than 33 μm/s, indicating outstandingly high sensitivity of the graphene microsheet-based coulometric measuring methodology.

The results shown in FIG. 2B also indicates a proportional relationship between the Faradaic current and the flow. Due to the atomic-thickness of the graphene microsheet and a negligible cross section area normal to the flow, the charge-transfer process on the microsheet is dominantly diffusion-governed. Under this regime, the Faradaic current can be described by ΔI_{F}=Fc°uₐᵥLh, wherein I_{F} is current on the electrode, n is the mole number of electrons transport during electrochemical reaction, F is the Faraday constant, c° is the bulk concentration of the redox species solution far away from the electrode, uₐᵥ is the average flow velocity inside channel, L is the width of channel, and h is the height of the channel. (Amatore, et al. “Theory and experiments of transport at channel microband electrodes under laminar flows. 1. Steady-state regimes at a single electrode.” Analytical Chemistry 79, 8502-8510 (2007).)

The setup of our experiment lead to c° of 4.4 e-12 mol/L. To compensate for the small deviation of response from the proportional relationship, particularly when the flow rate was low, the current-flow plot as a calibration chart was used to convert current response to flow.

Our approach was sensitive enough to respond to deviation of the flow in the experimental microfluidic system from an ideal system. The speed of the syringe pump that drove the PBS flowing through the microfluidic chip was set to triangle-wave-like ramps. The current response of real-time velocity ramp increased linearly from 0.33 to 6.57 mm/s in 20 s and followed the change of velocity from 0.06 to 1.68, with excellent repeatability. The relaxation of the response as well as the calibrated flow, as shown by FIG. 2c, took about 0.5 s when the flow decreases from 6.33 mm/s to zero. The fluctuation of the response may be understood by the noise that is generated by the syringe pump and the microfluidic channels. (Pennathur, “Flow control in microfluidics: are the workhorse flows adequate?” Lab on a Chip 8, 383 (2008); Lake, et al. “Low-cost feedback-controlled syringe pressure pumps for microfluidics applications.” PLoS One 12, (2017).)

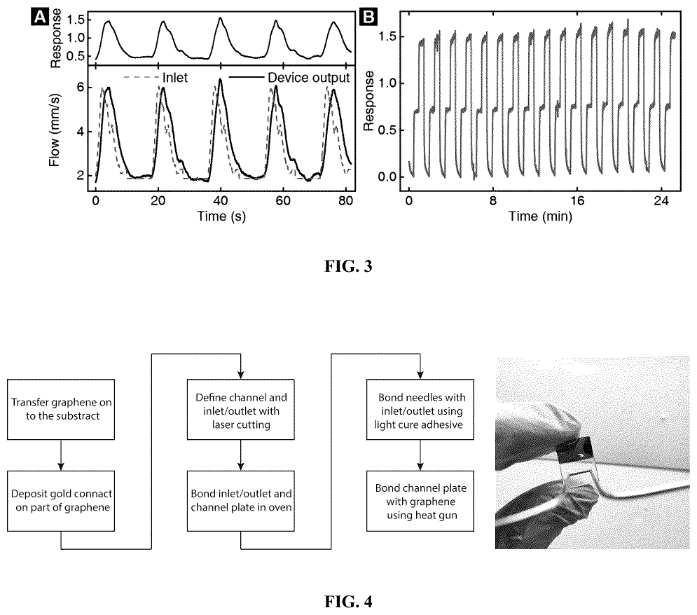

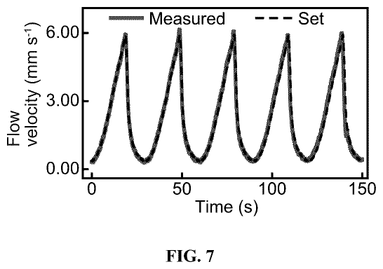

The methodology was used to interrogate the flow of full bovine blood. The response-flow relationship based on real-time charge transfer measurement (FIG. 6), as shown in FIG. 2B, agreed well with that for PBS, attributing to the ultra-thinness of the graphene microsheet that was minimally impacted by the non-specific components in the bovine blood. Using the current-flow plot as a calibration chart to convert current response to flow, the response to pump speed ramps (FIG. 7) and speeds that mimic the flows in human capillary (FIG. 1B) and arteriole (FIG. 3A) were measured. The speed waveform for arteriole was based on arteriole blood fluctuated with heartbeat. (Zhong, et al. “Noninvasive measurements and analysis of blood velocity profiles in human retinal vessels.” Investigative Ophthalmology & Visual Science 52, 4151-4157 (2011).)

The speed increased from 1.87 to 6.05 mm/s, right above the range of the capillary blood flow. The response and calibrate flow demonstrated excellent correlation with the speed of the syringe pump despite the minimal relaxation and fluctuation that can be attributed to the limit of the microfluidic system and the viscous effect of blood. Our results indicated that the coulometric interrogation strategy based on the graphene microelectrode was capable of characterizing a broad range of the flow of complex biofluids at high sensitivity.

Owing to the immunity of the graphene microsheet with planar configuration, the flow measurement using our approach was outstandingly robust. FIG. 3B shows results from long-term interrogation of blood flow that was changed in sequence of 0.033, 3 and 6 mm/s, with each lasting for 30 s, the device's current response maintained excellent stability.

### Examples

As a proof of concept, a graphene microsheet was prepared via chemical vapor deposition (CVD) on a copper catalytic substrate and transferred onto 50 μm-thick acrylic film using a low-contamination bubbling method. The graphene microsheet was then fabricated followed by the deposition of 60-nm thick Cr/Au electrodes for electrical contact. Microflow channel was defined on a 0.5 mm thick acrylic sheet using laser cutting and bonded to the graphene-PMMA film using dichloromethane (DCM) and 2-propanol (IPA).

An electrometer was used to measure the charge that transferred between the graphene electrode and biofluids with various flow rates. The Faradic current deducted from 20 seconds of charge-transferring measurement demonstrated proportional relationship with respect to the flow rate, the limit of detection of the flow was less than 33 μm/s and thus can be used to characterize the flow rate.

### Preparation of Graphene Sheets

Copper foil (99.8% purity) was loaded into a 4 in. quartz tube and annealed for 30 min at 1060° C. in an environment of ultrahigh purity (99.999%) hydrogen (flow rate=200 sccm) to eliminate the influence of oxide residues. Then low-pressure chemical vapor deposition was used with precursor of methane (flow rate=0.5 sccm, growth time of 20 min).

### Device Fabrication

400 nm layer of polymethyl methacrylate (950 PMMA A4, MICROCHEM) was spin coated on the graphene-copper growth substrate, followed by immersion in 1M NaOH solution with the graphene-copper growth substrate connected to the cathode of power supply to float off the PMMA-graphene film. The PMMA-graphene film was transferred onto a 0.5 mm thick PMMA film with PMMA contacting each other, and graphene became exposed to the environment. After being cut by blade, 5 nm/55 nm Cr/Au was deposited on part of graphene-PMMA film for electrical connection with the electrometer.

For the microfluidic device, fluid inlet/outlet PMMA plate and 500×500 μm PMMA micro-channel were defined by laser cutting and bonded by 80 vol % dichloromethane (DCM, Honeywell)-20 vol % 2-propanol (IPA, Fisher Chemical) mixed solution under 70° C. for 3 hours in oven. After bonding 304 stainless steel needles to the fluid inlet/outlet by light cure adhesive (Loctite AA 3951) and be exposed to light for 24 hours, the microfluidic plates with needles were cleaned by DI water with ultrasonic bath for 10 minutes to remove redundant adhesive. Then the DCM-IPA mixed solution was used again for bonding microfluidic plates and non-metal electrode part of graphene-PMMA film by baking for 90 seconds under 100° C. using a heat gun.

The fabrication process and the as-fabricated devices are shown in FIG. 4.

### Flow Rate Characterization

An electrometer (Keithley 6517a) with extremely low noise (virtually none) was used to measure the charge that transferred between the graphene and the solution because the charge-transfer rate (<pC/s) was very small at the graphene/solution interface. The setup of the measurement is shown in FIG. 1A. Before measurement, the noninverting input of electrometer was grounded while the inverting input was connected to a dissipating resistor for zero check. In order to start measurement, the inverting input was disconnected from a dissipating resistor and was connected to the Au/Cr contact. The biofluid flows through the microfluidic channel and cross on the graphene microsheet changed the diffusion layer thickness in the fluid, and the spontaneous Faradaic current variation was characteristic to the flow speed.

Real-time Faradaic charge transfer was measured in real-time for full phosphate buffered saline when the flow increased. The measurement had ultra-low noise with optimal signal-to-noise ratio of 45 dB. The slope was used to determine the Faradaic current response shown in FIG. 2B. The limit of detection of the flow was less than 33 μm/s, indicating outstandingly high sensitivity of the coulometric measuring methodology based on the graphene microsheet. The result also showed a proportional relationship between the Faradaic current and the flow. To compensate for the small deviation of response from the proportional relationship, particularly when the flow rate was low, the current-flow plot was used as a calibration chart to convert current response to flow. The current response of real-time velocity ramp increased linearly from 0.33 to 6.57 mm/s in 20 s and followed the change of velocity from 0.06 to 1.68, with excellent repeatability. The relaxation of the response as well as the calibrated flow, shown in FIG. 2C, took about 0.5 s when the flow decreased from 6.33 mm/s to zero.

### Step-by-Step Flow-Rate Measurement Using a Graphene Flow Sensor

An operational amplifier-based electrometer (1-fC charge measurement resolution, settling time <0.1 μs) was used to quantify the charge transfer of the graphene single microelectrode.

Prior to the measurement, the noninverting input of the amplifier was grounded while the inverting input was connected to a dissipative resistor. Then a picoliter/min syringe pump (Harvard Apparatus 70-3009) was used to drive the biofluid flow through the device microfluidic channel for more than 10 min in advance to ensure no that entrapment of bubbles was observed at the graphene device during the measurement.

To start the measurement, the inverting input was disconnected from the dissipative resistor and connected to the device Au/Cr lead, which linked the graphene single microelectrode to a virtual ground so that the charges that transferred from the flow to be interrogated into the graphene were stored in a feedback capacitor of the operational amplifier and quantified.

During the measurement, a program was used to acquire the flow-sensory charge signal from the coulometer through an IEEE-488 interface. The program performed time derivative of the measured charge to obtain the charge-transfer current and used a bandwidth variable Savitzky-Golay filter based on the least-squares polynomial method to smooth the current in real time. The measured electric current was translated to flow velocities in the program in real time by interpolating the current-flow data set of the device being tested.

After each measurement, PBS was infused by the syringe pump through the device at 5 mm s⁻¹ for 1 min. Then DI water was infused through the device at 5 cm s⁻¹ for 4 min, followed by infusing air with a speed of 10 cm s⁻¹ for 4 min to dry the device. The device was stored in an airtight, opaque container when not in use.

Materials, compositions, and components disclosed herein can be used for, can be used in conjunction with, can be used in preparation for, or are products of the disclosed methods and compositions. It is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compounds or compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Applicant's disclosure is described herein in preferred embodiments with reference to the FIG.s, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. An electrical nanodevice having a single microelectrode, comprising a monolayer graphene sheet serving as the single microelectrode disposed across a microfluidic channel.

2. The electrical nanodevice of claim 1, wherein the single microelectrode comprises a Cr/Au electrode deposited on a part of the monolayer graphene sheet for electrical connection.

3. The electrical nanodevice of claim 2, further comprising an operation amplifier having a feedback capacitor.

4. The electrical nanodevice of claim 3, wherein the microfluidic channel is defined on an acrylic sheet using laser cutting and bonded to the graphene sheet.

5. The electrical nanodevice of claim 3, wherein the monolayer graphene sheet is fabricated with a poly(methyl methacrylate) (PMMA) polymer layer.

6. The electrical nanodevice of claim 3, wherein the monolayer graphene sheet has a dimension in the range from about 1 μm×1 μm to about 1 mm×1 mm.

7. The electrical nanodevice of claim 3, wherein the microfluidic channel has a dimension in the range from about 1 μm×1 μm to about 1 mm×1 mm.

8. The electrical nanodevice of claim 3, wherein the electrical nanodevice does not comprise an external electrical supply.

9. The electrical nanodevice of claim 3, wherein the electrical nanodevice is biocompatible.

10. A microfluidic flow sensor comprising an electrical nanodevice of claim 1.

11. An implantable blood flow monitor comprising an electrical nanodevice of claim 1.

12. A method for measuring a microfluidic flow velocity, comprising measuring a hydrovoltaic current variation arising from contact electrification between a monolayer graphene sheet and a microfluidic flow.

13. The method of claim 12, wherein the hydrovoltaic current variation is measured in vivo via an implanted electrical nanodevice.

14. The method of claim 13, wherein the implanted electrical nanodevice does not comprise an external electrical supply.

15. The method of claim 12, wherein the microfluidic flow is that of a biofluid.

16. The method of claim 15, wherein the biofluid is whole blood.

17. The method of claim 12, capable of measuring the microfluidic flow velocity characterized by variations at the μm/s level.

18. The method of claim 13, capable of providing in vivo chronic body fluidic monitoring.

19. A method of fabricating an electrical nanodevice, comprising:
providing a monolayer graphene sheet on a substrate;
depositing a gold connection contact on a part of the monolayer graphene sheet;
providing a microfluidic channel with an inlet and an outlet; and
bonding the microfluidic channel with the monolayer graphene sheet.

20. The method of claim 19, wherein the monolayer graphene sheet is provided on a poly(methyl methacrylate) (PMMA) polymer layer.

21. (canceled)

22. (canceled)

23. (canceled)
